(19) United States
(12) Patent Application Publication
(10) Pub. No.: US 2004/0102530 A1
(43) Pub. Date: May 27, 2004

(54) Multistage compact fischer-tropsch reactor

(75) Inventors: Alessandro G. Borsa, Evergreen, CO (US); Nicholas E. Vanderborgh, Boulder, CO (US)

Correspondence Address: PATTON BOGGS, 1660 LINCOLN ST, SUITE 2050, DENVER, CO, 80264, US

(73) Assignee: Blue Star Sustainable Technologies Corporation, Arvada, CO (US)

(21) Appl. No.: 10/302,478

(22) Filed: Nov. 22, 2002

(51) Int. Cl. C07C 27/06; B01J 8/04

(52) U.S. Cl. 518/704; 422/191

(57) ABSTRACT

A multistage compact packed-bed Fischer-Tropsch reactor comprises a plurality of first-stage reaction tubes and a plurality of second-stage reaction tubes in a reaction-heat-exchange chamber of a reactor vessel. The interior space of each of the reaction tubes contains a packed bed of catalyst. The reactor vessel contains an interstage fluid process chamber and a heat exchanger for condensing hydrocarbon products and water. After passing through catalyst in the first-stage reaction tubes, a process gas stream is cooled by a heat exchanger within the reactor vessel to condense hydrocarbon products and water. The liquid hydrocarbons and water are removed from the reactor vessel. The product gas stream then enters the second-stage tubes in which it is preheated by transfer of heat from the first-stage reaction tubes. The reactor comprises an exit-fluid process chamber within the reactor vessel. After passing through the catalyst in the second-stage reaction tubes, the process gas stream is cooled by a second heat exchanger within the reactor vessel to condense hydrocarbon products and water out of the process gas stream. In the exit-fluid process chamber, liquid hydrocarbons and water are separated from the process gas stream.

### FIELD OF THE INVENTION

The invention relates to the production of liquid hydrocarbon fuels from natural gas, particularly to the production of high-quality liquid fuels utilizing a Fischer-Tropsch technique.

### BACKGROUND OF THE INVENTION

Large volumes of natural gas are available throughout the world. The conversion of natural gas to liquid fuels is an attractive route for utilizing the world's abundant gas reserves. There is a large demand for middle distillate fuels, and conversion of gas to liquid fuels circumvents the technical or economic infeasibility often associated with gas delivery through long distance pipelines, the large-scale production of gas-based chemicals, or the manufacture of liquefied natural gas (LNG).

Chemical engineering processes to upgrade gaseous hydrocarbons into transportable liquid fuels for use in engines were demonstrated 80 years ago. The technology advanced during World War II in both Germany and Japan as these countries sought secure sources of fuel to support their war efforts. During the 1950s, South Africa developed natural-gas-derived synthetic fuels to cushion the impact of potential petroleum shortages. For example, in 1955, Sasol Oil started utilizing a high-temperature Fischer-Tropsch process called Synthol, which produced approximately 150,000 barrels per day (“bpd”) of synthetic fuel (gasoline, diesel, and kerosene).

The Fischer-Tropsch reaction is a well-known mechanism for condensing a gaseous mixture of hydrogen and carbon monoxide, or synthesis gas (“syngas”), into a mixture of olefins, paraffins, and oxygenates in the presence of transition metal-based catalysts. Such catalysts may incorporate a first row non-noble metal such as iron, cobalt, or nickel as the predominant active site, along with a noble metal (ruthenium, platinum, rhenium), actinide (thorium), or alkali (lithium, sodium, potassium) promoter, optionally supported on a refractory, non-reducible oxide such as silica, alumina, or titania.

Conversion of synthesis gas by way of the Fischer-Tropsch reaction occurs as a result of a highly-exothermic chemical process, represented by:

CO+2H₂→CH₂+H₂O

The Fischer-Tropsch process can either be operated at high temperatures, in which case a light syncrude is produced, or at low temperatures, in which case a large fraction of the syncrude produced contains heavy, waxy hydrocarbons.

The first industrial atmospheric fixed-bed catalytic reactor, operated by Ruhrchemie in 1935, consisted of a box that was divided in sections by vertical metal sheets, and of horizontal cooling tubes crossing the sheets, the catalysts being loaded between sheets and tubes. Water cooling-medium in the tubes was maintained at the equilibrium vapor pressure of water at the desired cooling temperature. The enthalpy of reaction vaporized a corresponding amount of water, with the result that the temperature of the cooling medium was constant throughout the reactor, independent of the amount of locally recovered heat.

Fischer-Tropsch processes with iron or cobalt catalysts must accommodate specific catalyst characteristics. A cobalt-based catalyst is less susceptible to water inhibition than iron, so it can tolerate a small amount of water in the feed syngas. Nevertheless, water causes cobalt-catalyst degradation, and reaction conversion improves as water content decreases. Also, with cobalt, the selectivity is strongly dependent on the partial pressures of CO and H₂, and a H₂/CO ratio of 2:1 should be maintained in order to avoid excessive methane formation.

Performance of the Fischer-Tropsch synthesis depends strongly on reaction temperature. Increasing temperature favors selective methane formation and deposition of carbon and thereby deactivation of the catalyst, and reduces the average chain length of the product molecules. Because Fischer-Tropsch hydrogenation of carbon monoxide is strongly exothermic, an essentially isothermal process condition is not easily obtained; however, the better the isothermicity of a process, the higher the average temperature in a reactor can be. Even in a stable operating region, large temperature peaks in a packed-bed of catalyst are to be avoided as they may give rise to an undesired reduction of selectivity. Aside from the choice of tube diameter, catalyst particle size and gas velocity determine the effectiveness of radial heat transport and the homogeneity of the temperature in the bed. Heat conductivity as well as the heat transfer coefficient become higher with increasing Reynolds number; hence, heat removal becomes more effective with larger particles and at higher velocities. Nevertheless, considerations of catalyst-effectiveness and pressure drop limit an increase of particle size and velocity.

Conventional approaches to gas-to-liquid (“GTL”) projects seek cost savings through economies of scale and contemplate very large plants with production capacities in excess of 30,000 bpd. The giant-scale plants contemplated by the GTL industry today would be suitable only for a handful of fields in the entire world. Natural gas is a widely distributed resource. A large portion of this resource base is in continuous, tight, and basin-centered deposits, including coal seams, spread over large geographic areas. Deliverability rates from wells in these types of deposits are typically relatively low. In many instances, even in North America, the pipeline infrastructure is not nearby. The requirement for huge GTL plants consuming high volumes of natural gas runs counter-current to what is known about global natural gas resources.

Development of gas trapped in coal seams has become a major focus of the natural gas industry. Deliverability rates of gas from individual wells are generally low, often stabilizing in a range of from 200 MCFD to 500 MCFD (thousand cubic feet per day) only. In large gas fields, some areas contain gas with high levels of nitrogen or CO₂ that do not comply with quality specifications required by pipelines. In low-production oilfields and on offshore oil-well platforms with small quantities of associated or casing head gas, the gas produced in these fields is often flared because it is not economic to collect, process, and deliver into a natural-gas pipeline. In many rural locations throughout the world where ranching, agricultural, or other operations are being conducted at long distances from electrical power grids and liquid-fuel depots, it would be useful to convert local natural gas into electricity and liquid fuels. Even in areas close to usually dependable supplies of electricity and liquid fuel, the capability to convert relatively small amounts of natural gas into clean liquid fuel, heat, and electricity would be useful.

Common Fischer-Tropsch reactor designs include packed-bed, fluidized-bed and slurry reactors. A traditional packed-bed reactor includes a vertical reactor having tubes containing catalysts into which syngas is introduced. The syngas passes over the catalyst in the tubes and then exits the reactor. A fluid for removing or adding heat, such as boiling water, flows around the tubes. Some of these reactors have 10,000 or more tubes and might be 30 feet or 40 feet long. Frequently, it is difficult to load the tubes of a large vertical reactor design. It is often difficult or impossible to add an additional reactor or other substances at an intermediate point of the reaction process. Furthermore, it is generally difficult to vary the process gas flow rate and to remove reaction products during the reaction process. International Application PCT/US97/10732, having International Publication No. WO 98/04342, published 5 Feb. 1998, describes a fixed bed, catalytic, cross-flow reactor. As taught in WO 98/04342, a cooling medium flows in a longitudinal, horizontal direction within the interior of tubes located in a horizontal cylindrical reactor body. Sheets arranged transverse to the longitudinal tubes divide the reactor into separate zones. Each zone is loaded with catalyst on the outside and surrounding the tubes. Fluid feed stock enters the reactor at the top of a first reaction zone, flows transversely through the fixed bed of catalyst to the bottom of the zone, and then flows through a return conduit to the top of the next zone. Heat exchangers and separators located externally of the reactor body separate condensate (e.g., wax), if any, from the process fluid near the bottom of each reaction zone.

One of the main problems with Fischer-Tropsch reactors is the relatively low conversion per pass obtained in a reactor. In some processes, this problem is resolved by recycling un-reacted syngas from the product stream, which has beneficial effects on heat removal and reactor efficiency. This technique is described in U.S. Pat. No. 4,587,008, issued May 6, 1986 to Minderhoud et al., disclosing a two-step process wherein C9+ hydrocarbons are prepared from C4− hydrocarbons by steam reforming followed by Fischer-Tropsch synthesis over a cobalt catalyst, and in which yields of C9+ hydrocarbons are increased by recycling a gaseous fraction comprising unconverted H₂ and CO, as well as C8− hydrocarbon by-products and steam, to the steam reformer.

Due to economic and size considerations, it would be unfeasible for a small-scale Fischer-Tropsch system to include an oxygen separation plant supplying it with oxygen. Therefore, in a small-scale Fischer-Tropsch system, air is used in reforming of natural gas to make syngas. The use of air, however, introduces a considerable amount of nitrogen into the syngas. In cases where the syngas stream contains a substantial amount of inerts, such as syngas produced by partial oxidation with air, recycling un-reacted gases becomes practically impossible to accomplish.

### SUMMARY OF THE INVENTION

Embodiments in accordance with the present invention help to solve some of the problems mentioned above. In one aspect, a method and a multistage compact Fischer-Tropsch reactor in accordance with the invention provide a plurality of packed-bed catalytic reaction stages enclosed in a reactor vessel for converting syngas into medium-weight hydrocarbon fuel. In another aspect, a heat exchanger condenses product hydrocarbons and water between reaction stages. In another aspect, liquid hydrocarbons and liquid water are removed from the reactor vessel between reaction stages. Removal of water from the process gas stream reduces degradation of catalyst by water. Removal of product hydrocarbons and water from the process gas stream also increases the partial pressures of CO and H₂, thereby increasing selectivity and yields of the Fischer-Tropsch reaction. In another aspect, a portion of the exothermic heat of reaction is used within the reactor vessel to preheat a process gas stream before it enters a reaction stage. In another aspect, a plurality of unit operations are integrated in the reactor vessel, thereby decreasing capital equipment costs, decreasing space requirements, and increasing thermal efficiency compared to conventional Fischer-Tropsch reaction systems. Integrated unit operations contained in a single reactor vessel include catalytic reaction, preheating of process gas streams, cooling of process gas streams, condensation of reaction products, and gas-liquid separation. In another aspect, a system in accordance with the invention provides a plurality of chambers in a single reactor vessel that are practically separate temperature zones. Good control of reaction temperature in the reactor vessel provides good product selectivity and reaction yields.

In one aspect, a reactor in accordance with the invention comprises a reactor vessel, a first-stage tube disposed in the reactor vessel, and a second-stage tube disposed in the reactor vessel. In another aspect, the reactor vessel contains an interstage fluid process chamber, a first heat exchanger, and a liquid-removal outlet in the interstage fluid process chamber. In another aspect, the heat exchanger is disposed in the interstage fluid process chamber. In another aspect, a baffle is disposed in the interstage fluid process chamber. In still another aspect, the interstage fluid process chamber comprises an interstage syngas inlet. In still another aspect, a reactor in accordance with the invention comprises an exit-fluid process chamber disposed in the reactor vessel, and a liquid-removal outlet in the exit-fluid process chamber. In still another aspect, a reactor comprises a process gas outlet in the exit-fluid process chamber. In another aspect, a reactor comprises a second heat exchanger disposed in the exit-fluid process chamber for condensing hydrocarbons and water from a process gas. In another aspect, a reactor comprises a baffle disposed in the exit fluid process chamber.

In another aspect, a reactor comprises a reaction-heat-exchange chamber disposed in the reactor vessel, wherein a first-stage reaction portion of the first-stage tube is located in the reaction-heat-exchange chamber, and a second-stage reaction portion of the second-stage tube is located in the reaction-heat-exchange chamber. In still another aspect, a reactor comprises a packed bed of Fischer-Tropsch catalyst disposed within the first-stage reaction portion of the first-stage tube, and a packed bed of Fischer-Tropsch catalyst disposed within the second-stage reaction portion of the second-stage tube. In another aspect, a reactor comprises a fluid heat-exchange medium disposed in the reaction-heat-exchange chamber, the heat-exchange medium being in thermal contact with an outer surface of the reaction portions. In another aspect, the fluid heat-exchange medium is selected from a group consisting of water and thermal oil. In another aspect, a reactor comprises a pressure controller for maintaining a pressure in the reaction-heat-exchange chamber exterior to the tubes.

In still another aspect, a reactor comprises an interstage heat-exchange chamber disposed in the reactor vessel, wherein a first-stage outlet portion of the first-stage tube is located in the interstage heat-exchange chamber, and a second-stage inlet portion of the second-stage tube is located in the interstage heat-exchange chamber. In another aspect, a reactor comprises a heat-exchange medium disposed in the interstage heat-exchange chamber, the heat-exchange medium in thermal contact with an outside surface of the outlet portion and with an outside surface of the inlet portion. In another aspect, the fluid heat-exchange medium is water or thermal oil. In another aspect, the first-stage outlet portion and the second-stage inlet portion do not contain catalyst. This is because the inlet portions and the outlet portions of a reaction tube are outside of the reaction-heat-exchange chamber, which is maintained at a desired reaction temperature to achieve good selectivity and to avoid undesired reaction products. In another aspect, to achieve good thermal conductivity within the reaction tubes and a good heat transfer rate between the interior reaction space of the reaction tubes and a heat transfer medium external to the reaction tubes, the first-stage outlet portion and the second-stage inlet portion contain blank packing. Preferably, to ensure that the process gas stream is preheated to a desired reaction temperature before it reaches catalyst and starts reacting, the first-stage tube comprises a first-stage inlet portion disposed at least partly in the reaction-heat-exchange chamber, wherein the first-stage inlet portion does not contain catalyst. Preferably, to improve thermal conductivity and heat transfer rate, the first-stage inlet portion comprises blank packing.

In still another aspect, a reactor in accordance with the invention comprises a feedstock heat-exchange chamber disposed in the reactor vessel, and in which the first-stage tube comprises a first-stage inlet in fluidic communication with the feedstock heat-exchange chamber, and the feedstock heat-exchange chamber comprises at least part of a second-stage outlet portion of the second-stage tube. This provides transfer of heat from the second-stage outlet portion to the process gas in the feedstock heat-exchange chamber as it enters the first-stage reaction tubes.

In one aspect, a method of conducting a Fischer-Tropsch reaction in a multistage compact packed-bed reactor comprises flowing process gas containing inlet syngas through a first catalyst bed, then first-stage-cooling the process gas within the reactor vessel to condense hydrocarbons and water from partially reacted process gas, then flowing the partially reacted process gas into a second catalyst bed, and then second-stage-cooling the process gas within the reactor vessel to condense hydrocarbons and water from the process gas. In another aspect, a method further comprises removing liquid hydrocarbons and liquid water from the reactor vessel after the first-stage cooling. In another aspect, the first-stage cooling comprises contacting an exterior surface of a first-stage outlet portion of the first-stage reaction tube with a heat-exchange medium. In another aspect, the first-stage cooling comprises flowing the process gas through a heat exchanger disposed in an interstage fluid processing chamber. Preferably, the first-stage cooling and the second-stage cooling are conducted at a temperature in a range of about from 20° C. to 40° C.

In another aspect, a method in accordance with the invention comprises removing liquid hydrocarbons and liquid water from the reactor vessel after the second-stage cooling. In another aspect, a method comprises maintaining a pressure in the catalyst beds, preferably in a range of about from 10 atmospheres to 20 atmospheres. In another aspect, a method in accordance with the invention comprises maintaining a temperature of the first catalyst bed and the second catalyst bed, preferably in a range of about from 150° C. to 280° C.

In another aspect, maintaining a temperature of the catalyst beds comprises contacting an exterior surface of the reaction tubes with a high-temperature heat-exchange medium, for example, with a thermal oil. In another aspect, maintaining a temperature of the catalyst beds comprises providing liquid water in a reaction-heat-exchange chamber and maintaining a pressure in the reaction-heat-exchange chamber such that the liquid water boils at a desired reaction temperature. Preferably, a method in accordance with the invention comprises first-stage-preheating the process gas before flowing the process gas through the first catalyst bed. In one aspect, the first-stage-preheating comprises contacting an exterior surface of a first-stage inlet portion of the first-stage reaction tube with the high-temperature heat-exchange medium, thereby transferring internal system heat to the first-stage inlet portion. In another aspect, a method in accordance with the invention further comprises second-stage-preheating the process gas before flowing the process gas through the second catalyst bed. In another aspect, second-stage-preheating comprises contacting an exterior surface of a first-stage outlet portion of the first-stage reaction tube with a heat-exchange medium, and contacting an exterior surface of a second-stage inlet portion of a second-stage reaction tube with the heat-exchange medium, thereby transferring internal system heat from the first-stage reaction tube to the second-stage reaction tube.

In another aspect, an embodiment in accordance with the invention provides a multistage compact chemical reactor, comprising a reactor vessel, a first-stage tube disposed in the reactor vessel, a second-stage tube disposed in the reactor vessel, a heat exchanger disposed in the reactor vessel, an interstage fluid process chamber disposed in the reactor vessel, and a fluid removal outlet in the interstage fluid process chamber. In another aspect, a multistage compact chemical reactor in accordance with the invention further comprises an exit-fluid process chamber disposed in the reactor vessel. In still another aspect, a multistage compact chemical reactor further comprises an interstage heat-exchange chamber disposed in the reactor vessel.

### BRIEF DESCRIPTION OF THE DRAWINGS

A more complete understanding of the invention may be obtained by reference to the drawings, in which:

FIG. 1 depicts a process flow diagram of a generalized small-scale process for converting light hydrocarbons to high-quality liquid hydrocarbon fuel using a Fischer-Tropsch reactor in accordance with the invention;

FIG. 2 depicts in schematic form a cross-section of a preferred embodiment of a multistage compact packed-bed Fischer-Tropsch reactor in accordance with the invention having two reaction stages;

FIG. 3 depicts in schematic form a cross-section of a multistage compact packed-bed Fischer-Tropsch reactor in accordance with the invention having four reaction stages; and

FIG. 4 depicts in schematic form a cross-section of a preferred embodiment of a multistage compact packed-bed Fischer-Tropsch reactor in accordance with the invention having four reaction stages.

### DETAILED DESCRIPTION OF THE PREFERRED EMBODIMENT

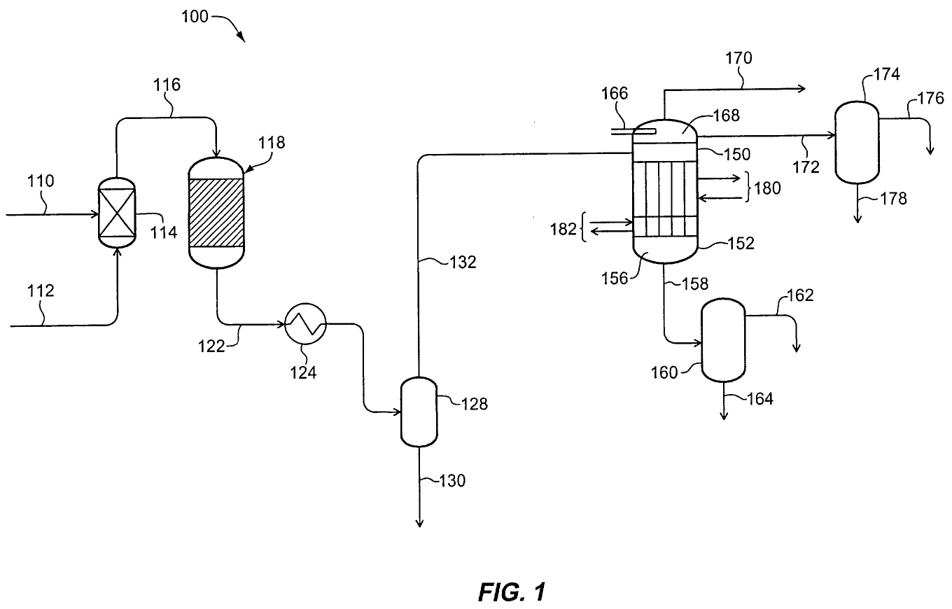

The invention is described herein with reference to FIGS. 1-4. It should be understood that the structures and systems depicted in schematic form in FIGS. 1-4 are used to explain the invention and are not precise depictions of actual structures and systems in accordance with the invention. Furthermore, the preferred embodiments described herein are exemplary and are not intended to limit the scope of the invention, which is defined in the claims below.

The term “light hydrocarbons” and related terms in the specification refer generally to hydrocarbons having molecule chain lengths of eight carbons (“C8”) or less. The term “medium-weight hydrocarbons” and related terms generally refer to hydrocarbons having chain lengths in a range of C9 to C35. The term “heavy hydrocarbons” generally refers to hydrocarbons having chain lengths greater than C35. Preferred medium-weight liquid hydrocarbon product fuels produced in reactors and methods in preferred embodiments in accordance with the invention include diesel fuel, having a predominant chain length of about C19.

A multistage packed-bed compact Fischer-Tropsch reactor described in the specification comprises a cylindrically-shaped reactor vessel and longitudinal tube bundle oriented vertically. Terms of orientation, such as “above”, “below”, “top”, “bottom”, and similar terms, are used in the specification in a manner consistent with the accompanying drawings. It is understood, however, that the geometry and spatial orientation of elements of the invention may vary from those depicted in the specification without departing from the scope of the invention. The terms “lower” and “higher” also designate relative positions of reaction stages in sequence in a multistage reactor, rather than having spatial significance.

The terms “process stream”, “process gas”, and related terms in the specification are used generally to designate the gaseous process flow stream entering, flowing through, and exiting a multistage compact Fischer-Tropsch reactor in accordance with the invention. It is clear that the composition of the gaseous process stream changes in the reactor and is, therefore, different at different points in the reactor. For example, the process stream entering the reactor contains relatively high levels of syngas, whereas the process stream entering an interstage heat-exchange chamber comprises reduced levels of syngas, and correspondingly high levels of water and hydrocarbon reaction products.

The term “heat exchanger” is used broadly in the specification to refer to any combination of structures and fluids by which heat is transferred from one fluid to another. The integrated heat exchangers in accordance with the invention are located within a reactor vessel and are utilized to effect heat exchange within the reactor vessel, including condensation-cooling, preheating of reactant syngas, and maintaining of a desired reaction temperature in catalyst beds. For example, a combination of an interstage heat-exchange chamber, an outlet portion of a first-stage reaction tube, an inlet portion of a second-stage reaction tube, and low-temperature heat-exchange medium functions as a heat exchanger exchanging heat between relatively hot process gas in the outlet portion of a first-stage reaction tube to the heat-exchange medium and to the relatively cool process gas in the inlet portion of a second-stage reaction tube. The term “heat exchanger” also includes heat-exchange coils, shell-and-tube exchangers, and others. The term “cooling coil” is used broadly in the specification to include heat exchangers with fins, as well as smooth coils.

FIG. 1 depicts a process flow diagram of a generalized small-scale process 100 for converting light hydrocarbons, such as methane and natural gas, to 10 barrels per day (“bbl/d”) high-quality liquid hydrocarbon fuel using a Fischer-Tropsch reactor in accordance with the invention. Light-hydrocarbon compressed gas stream 110, typically comprising methane, flows at a flow rate of about 150 standard cubic feet per minute (“SCFM”) at a pressure of about 20 atm into gas-mixing vessel 114, in which it is mixed with oxygen-containing gas stream 112, typically compressed air flowing at a flow rate of about 550 SCFM at about 20 atm pressure. Mixed gas stream 116 flows into a catalytic partial oxidation (“CPOX”) reactor 118 at a flow rate of about 700 SCFM at 20 atm pressure. CPOX reactor 118 converts from about 80 percent to 95 percent of the light hydrocarbons of gas stream 116 to synthesis gas (“syngas”), comprising H₂-gas and CO, typically with a H₂:CO ratio in a range of about 1.9 to 2.3, preferably about 2:1. In addition to nitrogen and syngas constituents hydrogen and carbon monoxide, syngas stream 122 exiting from CPOX reactor 118 typically comprises carbon dioxide, water, and unreacted natural gas or methane. Syngas stream 122 typically comprises about 40 percent to 50 percent nitrogen by volume. CPOX reactor 118 typically operates at a temperature of about from 700° C. to 1000° C., preferably at 20 atm pressure. Syngas stream 122 flows through cooling heat exchanger 124 and separator 128 to condense and separate water out of the gas stream. Condensed water exits separator 128 in exit stream 130, and dried syngas stream 132 flows from separator 128 into a multistage compact packed-bed Fischer-Tropsch reactor 150, which is fabricated and operated in accordance with the invention. Gaseous syngas inlet stream 132 entering reactor 150 typically has a temperature in a range of about from 25° C. to 300° C., preferably about 200° C., and pressure in a range of about from 10 atm to 40 atm, preferably about 20 atm. As depicted schematically in FIG. 1, multistage packed-bed Fischer-Tropsch reactor 150 comprises a cylindrical reactor vessel 152 comprising two reaction stages. Both stages together contain about 1000 kg of Fischer-Tropsch catalyst having a volume of about 1000 liters. After a first stage reaction, medium-weight and heavy (if present) hydrocarbons and water are condensed out of the process gas. Preferably, the liquid condensate contains virtually no heavy hydrocarbons. The liquid hydrocarbon product and water are collected in an interstage fluid process chamber 156 and flow in outlet liquid stream 158 to phase separator 160. In phase separator 160, liquid medium-weight product hydrocarbons (in stream 162) at a flow rate of about 6 bbl/d, typically in the C9 to C35 range, are separated from condensed liquid water at a flow rate of about 12 bbl/d (stream 164) (and from heavy hydrocarbons, if present). The first-stage conversion of syngas is typically about 40 percent. After the process gas passes through the second reaction stage in reactor 150, the process gas is cooled by heat exchanger 166 in an exit-fluid process chamber 168. As a result, product liquid hydrocarbon and water are condensed out of the process gas. Light hydrocarbons, unreacted syngas, nitrogen, and other relatively volatile compounds exit reactor 150 in outlet gas stream 170. Outlet liquid stream 172 flows from exit-fluid process chamber 168 to phase separator 174, in which medium-weight liquid hydrocarbon product with a flow rate of about 4 bbl/d (stream 176) is separated from water having a flow rate of about 8 bbl/d (stream 178) and heavy hydrocarbons (if present). The second-stage conversion of syngas is approximately 40 percent of the unreacted syngas entering the second stage. Thus, the overall conversion of a two-stage process in accordance with the invention is typically about 60 percent. In preferred embodiments, overall output is increased by adding fresh syngas to the gaseous process stream between stages. A high-temperature heat-exchange medium (arrows 180) functions simultaneously to maintain a desired temperature in the reaction stages of the highly exothermic Fischer-Tropsch reaction and to preheat reactants. A relatively low-temperature heat-exchange medium (arrows 182) functions to cool high-temperature process gas to effect condensation, and simultaneously preheats second-stage reactants. This enhances the thermal efficiency of the process. Also, preheating the process gas before it enters the catalyst bed enhances selectivity and yield of the Fischer-Tropsch reaction.

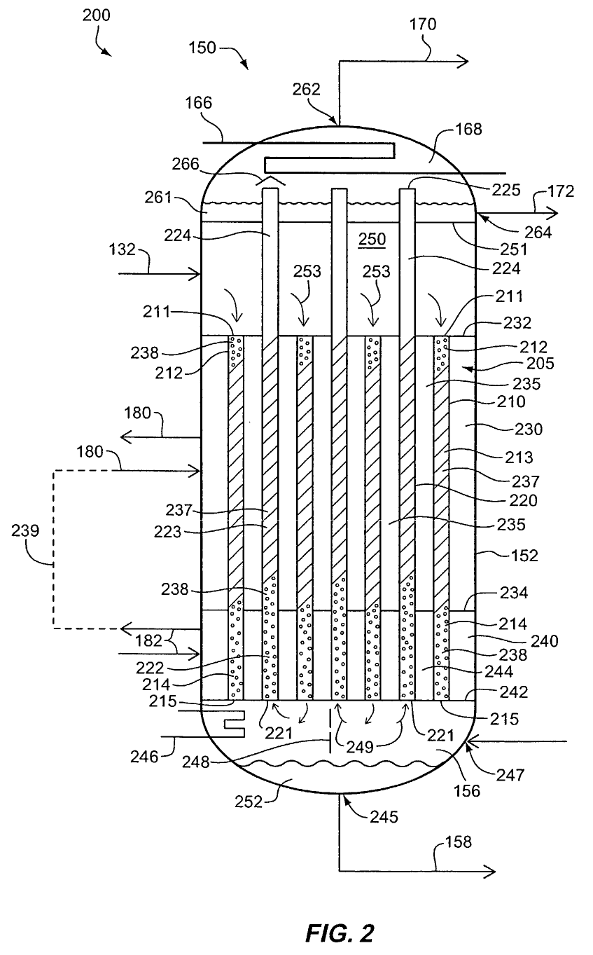

FIG. 2 depicts in schematic form a cross-section 200 of a preferred embodiment of a multistage compact packed-bed Fischer-Tropsch reactor in accordance with the invention. For the sake of clarity, reference numerals in FIG. 2 correspond with reference numerals of FIG. 1, where appropriate. Reactor 150 comprises a reactor pressure vessel 152, fabricated by methods known in the art to withstand operating pressures in excess of 40 atm. Reactor 150 further comprises a tube bundle 205, disposed within vessel 152. Tube bundle 205 includes a plurality of first-stage tubes 210. Each of first-stage tubes 210 comprises a first-stage inlet 211, a first-stage inlet portion 212, a first-stage reaction portion 213, a first-stage outlet portion 214, and a first-stage outlet 215. Tube bundle 205 further includes a plurality of second-stage tubes 220, each of which includes a second-stage inlet 221, a second-stage inlet portion 222, a second-stage reaction portion 223, a second-stage outlet portion 224, and a second-stage outlet 225. Each of first-stage reaction tubes 210 defines an interior reaction space, which includes the interior of reaction portion 213, where a packed bed of catalyst particles is disposed during operation to catalyze a first-stage Fischer-Tropsch reaction. Broadly viewed, the interior reaction space of reaction tubes 210 also includes first-stage inlet portion 212 and first-stage outlet portion 214. Similarly, each of second-stage reaction tubes 220 defines an interior reaction space, which includes the interior of second-stage reaction portion 223, where a packed bed of catalyst particles is disposed during operation to catalyze a first-stage Fischer-Tropsch reaction. The interior reaction space of reaction tubes 220 also includes second-stage inlet portion 222 and second-stage outlet portion 224. A cobalt-based Fischer-Tropsch catalyst is preferred for the conversion of synthesis gas to liquid fuels due to the high activity and the long life of this type of catalyst. Co-pending and commonly-owned U.S. application Ser. No. 10/083,176, filed Feb. 26, 2002, which is hereby incorporated by reference, teaches preferred cobalt-based Fischer-Tropsch catalysts utilized in packed catalyst beds in a small-scale Fischer-Tropsch process.

Reactor 150 further comprises an interstage fluid process chamber 156 enclosed within the bottom of reactor vessel 152. First-stage tube outlet 215 and second-stage tube inlet 221 are disposed in interstage fluid process chamber 156. Reactor 150 further comprises a reaction-heat-exchange chamber 230 enclosed within reaction vessel 152. Reaction-heat-exchange chamber 230 is defined by tube plate 232, above, and tube plate 234, below. Typically, two circle-shaped plates 232, 234 are oriented perpendicular to the longitudinal orientation of tubes 210, 220. Typically, the circumferential edges of two plates 232, 234 are welded to the inside walls of reactor vessel 152. Welding or other suitable technique seals the plurality of holes through tube plates 232, 234 through which tubes 210, 220 pass. During operation, the open spaces 235 in chamber 230 external to reaction tubes 210, 220 are filled with a high-temperature heat-exchange medium, typically, thermal oil or water. A Fischer-Tropsch reaction occurs in catalyst beds 237 inside reaction portions 213, 223 of tubes 210, 220, respectively. Since the Fischer-Tropsch reaction is highly exothermic, the high-temperature heat-exchange medium functions mainly as a cooling medium, and heat is continuously removed from chamber 230 by sending cool heat-exchange medium into the chamber and removing hot heat-exchange medium, as indicated by heat-exchange arrows 180. The catalytic Fischer-Tropsch reaction inside tubes 210, 220 is typically conducted in a range of about from 150° C. to 280° C., preferably at about 220° C. The temperature of the heat-exchange medium is regulated using techniques known in the art to maintain a stable reaction temperature in catalyst beds 237. The high-temperature heat-exchange medium within reaction-heat-exchange chamber 230 also preheats inlet gas in first-stage inlet portions 212, which are located at least partly within chamber 230 and which typically contain blank, chemically inactive packing 238, instead of catalyst. Thus, some of the exothermic heat of reaction produced in reaction tubes 210, 220 is transferred within reactor vessel 152 to preheat inlet syngas before it enters the high-temperature catalyst. This enhances the thermal efficiency of the reactor. Also, preheating the process gas before it enters the catalyst bed enhances selectivity and yield of the Fischer-Tropsch reaction.

Reactor 150 also includes an interstage heat-exchange chamber 240 enclosed within reactor vessel 152, and disposed between reaction-heat-exchange chamber 230 and interstage fluid process chamber 156. Interstage heat-exchange chamber 240 is defined by tube plate 234, above, and bottom end-plate 242. During operation, the open spaces 244 in chamber 240 external to tubes 210, 220 are filled with a low-temperature heat-exchange medium, typically thermal oil or water. The heat-exchange medium is circulated into and out of interstage heat-exchange chamber 240, indicated by arrows 182. In preferred embodiments, heated heat-exchange medium from interstage heat-exchange chamber 240 is used to preheat the high-temperature heat-exchange medium entering reaction-heat-exchange chamber 205, as indicated by dashed line 239. Preferably, heated heat-exchange medium from interstage heat-exchange chamber 240 is fed directly into reaction-heat-exchange chamber 230. Preheating the high-temperature heat-exchange medium used in reaction-heat-exchange chamber 230 increases the total amount of high-quality heat produced by reactor 156 and available for various heating uses, such as for air-conditioning. The temperature of the low-temperature heat-exchange medium in chamber 240 is regulated to cool process gases flowing out of catalyst beds 237 through first-stage outlet portions 214 of reaction tubes 210 in order to condense medium-weight hydrocarbons and water (and heavy hydrocarbons, if present) out of the process stream. The liquid water and liquid product hydrocarbons 252 are collected in interstage fluid process chamber 156 and removed in liquid stream 158 through liquid removal outlet 245. Optionally, cold-water cooling coils or other cooling heat exchanger 246 is used to condense and collect water and liquid hydrocarbons in interstage fluid process chamber 156. Liquid stream 158 flows to phase-separator 160 (see FIG. 1). Interstage fluid process chamber 156 also includes syngas makeup inlet 247 for adding syngas to the process stream as it flows into second-stage inlets 221. Optionally, interstage fluid process chamber 156 contains a baffle system 248, which is useful for blocking liquid particles and separating them from a gaseous process stream, and also for directing a gaseous process stream (arrows 249) into inlets 221 of second-stage tubes 220.

Second-stage inlet portions 222 of tubes 220 preferably contain blank packing 238 instead of catalyst. Process gases flowing through second-stage inlet portions 222 are preheated in interstage heat-exchange chamber 240. In this manner, process heat is internally transferred through low-temperature heat-exchange medium in chamber 240 from first-stage outlet portions 214 to second-stage inlet portions 222. Typically, second-stage inlet portions 222 containing blank packing extend partly into reaction-heat-exchange chamber 230. Process gas entering packed catalyst beds 237 in second-stage reaction portions 223 is preheated by the high-temperature heat-exchange medium in chamber 230. In this manner, the exothermic heat of the Fischer-Tropsch reaction is internally transferred from first-stage reaction portions 213 to the incoming second-stage process gas. An inlet portion without catalyst allows the process gas in an inlet portion to be preheated to a desired reaction temperature before passing through catalyst, where it starts to react. Preheating of the process gas before catalytic reaction enhances selectivity and yield of the reaction. Compared to an empty space, blank packing used in inlet and outlet portions of reaction tubes provides enhanced heat conductivity within the reaction tubes and enhance heat transfer between the interior of the reaction tubes and a heat exchange medium.

Reactor 150 further includes a feedstock heat-exchange chamber 250 enclosed within reactor vessel 152 and located above reaction-heat-exchange chamber 230. Feedstock heat-exchange chamber 250 is defined by end plate 251, above, and tube plate 232, below. As depicted in FIG. 2, second-stage tubes 220 pass through feedstock chamber 250, and there is no direct fluid communication between the interior of chamber 250 and the interior reaction space of tubes 220. The open spaces in feedstock heat-exchange chamber 250 external to reaction tubes 220 are generally filled with syngas feedstock from stream 132 entering inlet chamber 250. Fresh feedstock syngas in inlet chamber 250 is preheated through heat transfer from hot second-stage outlet portions 224 of hot reaction tubes 220. Conversely, process gas flowing through second-stage outlet portions 224 is cooled. In this manner, some of the exothermic heat of the Fischer-Tropsch reaction is internally transferred within reactor vessel 152. Preheated syngas (arrows 253) enters reaction tubes 210 through first-stage inlets 211, which are in fluidic communication with feedstock heat-exchange chamber 250. Typically, first-stage inlet portions 212 contain blank packing 238 instead of catalyst. As a result, the incoming syngas is further preheated before reaching catalyst beds 237. This enhances thermal efficiency of the reactor and enhances selectivity and yield of the Fischer-Tropsch reaction.

Reactor 150 includes exit-fluid process chamber 168, enclosed within reactor vessel 152. Chamber 168 is defined by end plate 251, below, and the top, convex wall of reactor vessel 152. After process gas passes through the second catalytic reaction stage of catalyst beds 237 in second-stage reaction portions 223, and is precooled in feedstock heat-exchange chamber 250, the process gas is further cooled by heat exchanger 166 in exit-fluid process chamber 168. As a result, liquid hydrocarbon products and water 261 are condensed out of the process gas. Light hydrocarbons, unreacted syngas, nitrogen and other relatively volatile compounds exit reactor 150 through gas outlet 262 in outlet gas stream 170. Liquid hydrocarbon products and water 261 exit through liquid removal outlet 264 in outlet liquid stream 172, which flows to a phase-separator 174 (see FIG. 1). Heat exchanger 166 is regulated so that the condensed liquids 261 and the remaining outlet gases have a temperature in a range of about from 20° C. to 40° C. Preferably, reactor 150 comprises tube-caps or baffles 266 in exit-fluid process chamber 168 to inhibit undesired entry of condensed liquids into reaction tubes 220. Baffles also function to direct the flow of gases across heat exchanger 166 to improve heat transfer.

End plate 251, tube plates 232, 234, and end plate 242 are fabricated by techniques known in the art so that the space within each of chambers 156, 230, 240, 250, and 168 contained within reactor vessel 152 is fluidically isolated from the respective space in other chambers, except for the flow of the process gas stream through reaction tubes 210, 220. Each of chambers 156, 230, 240, 250, and 168 functions practically as an independent temperature zone. During operation, fluid in the open space in each chamber, external to reaction tubes 210, 220, is maintained practically isothermally at a temperature different from the temperature in the other chambers.

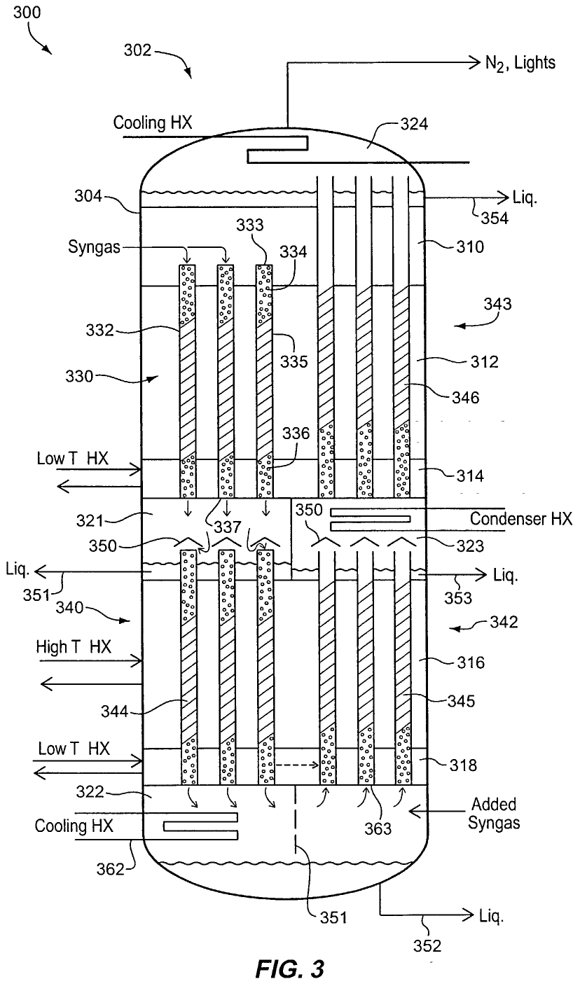

FIG. 3 depicts in schematic form a cross-section 300 of a multistage compact packed-bed Fischer-Tropsch reactor 302 in accordance with the invention. Reactor 302 comprises four catalytic reaction stages, and provides condensation and removal of hydrocarbon products and water after each stage. Reactor 302 comprises structural and operational elements similar to those of reactor 150, described with reference to FIG. 2. Reactor 302 includes reactor vessel 304. Reactor 302 comprises feedstock heat-exchange chamber 310, a reaction-heat-exchange chamber 312, an interstage heat-exchange chamber 314, a reaction-heat-exchange chamber 316, an interstage heat-exchange chamber 318, a first interstage fluid process chamber 321, a second interstage fluid process chamber 322, a third interstage fluid process chamber 323, and an exit-fluid process chamber 324, all enclosed within reactor vessel 304. Chambers 310, 312, 314, 316, 318, 321, 322, 323, and 324 are fluidically separated from each other, except for passage of the gaseous process strain through reaction tubes between chambers. Also, chambers 310, 312, 314, 316, 318, 321, 322, 323, and 324 function practically as separate temperature zones. During operation, reaction-heat-exchange chambers 312, 316 contain a high-temperature heat-exchange medium, and interstage heat-exchange chambers 314, 318 contain a low-temperature heat-exchange medium, as described above with reference to FIG. 2. Reactor 302 comprises first-stage tube bundle 330 comprising reaction tubes 332, each of tubes 332 having a first-stage inlet 333, a first-stage inlet portion 334, a first-stage reaction portion 335, a first-stage outlet portion 336, and a first-stage outlet 337. Reactor 302 further comprises second-stage tube bundle 340, third-stage tube bundle 342, and fourth-stage tube bundle 343, each comprising reaction tubes 344, 345, 346, respectively, similar to those already described herein. Syngas feedstock enters first-stage tubes 332. A partially reacted process stream gas is cooled to condense liquid hydrocarbon products and water, and condensate is removed in liquid outlet stream 351. Similarly, syngas reactants in the process stream flow through the second, third, and fourth stages of reactor 302, and liquid product hydrocarbons and water are removed from the reactor in liquid streams 352, 353, and 354, respectively. As depicted in FIG. 3, tube caps 350 inhibit undesired entry of liquid condensed out of a reaction stage into reaction tubes of the next-higher stage. Baffles 351 in interstage fluid process chamber 322 also inhibit the entertainment of liquid into the process stream, as well as direct the process stream in a desired manner to optional cooling heat exchanger 362 and to third-stage inlets 363.

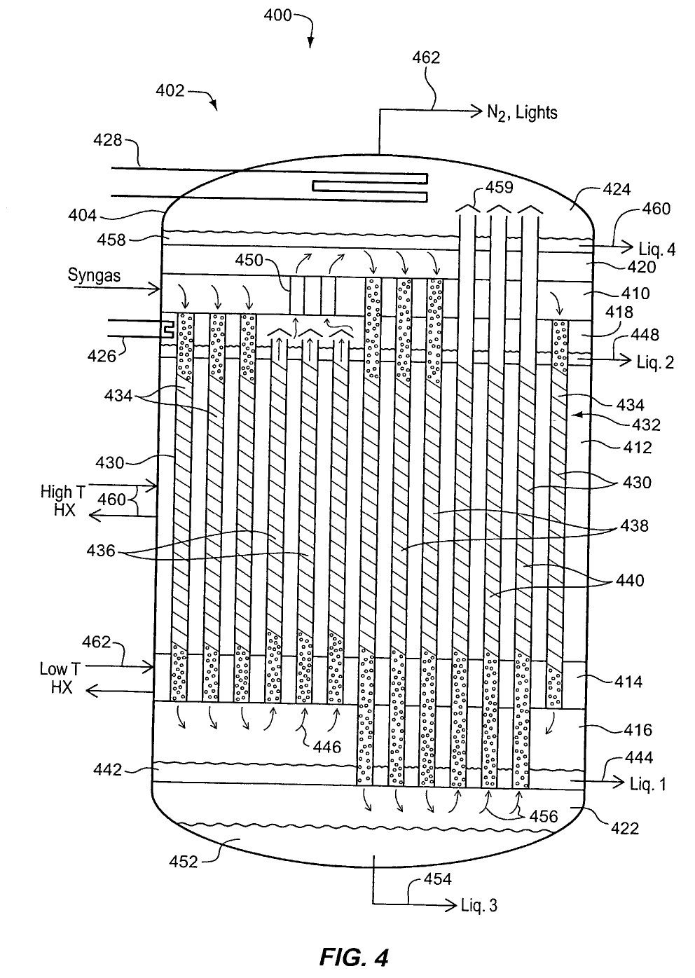

FIG. 4 depicts in schematic form a cross-section 400 of a preferred multistage compact packed-bed Fischer-Tropsch reactor 402 in accordance with the invention. Reactor 402 comprises four catalytic reaction stages, and provides condensation and removal of liquid hydrocarbon products and liquid water after each stage. Reactor 402 comprises structural and operational elements similar to those of reactor 150, described with reference to FIG. 2. Reactor 402 includes reactor vessel 404. Reactor 402 comprises feedstock heat-exchange chamber 410, a reaction-heat-exchange chamber 412, an interstage heat-exchange chamber 414, a first interstage fluid process chamber 416, a second interstage fluid process chamber 418, a dried-gas chamber 420, a third interstage fluid process chamber 422, and an exit-fluid process chamber 424, all enclosed within reactor vessel 404. Second interstage fluid process chamber 418 generally contains a heat exchanger 426 for cooling process gas to condense water and heavy and medium-weight hydrocarbons out of the gas. Similarly, exit-fluid process chamber 424 contains heat-exchanger 428 for cooling process gas to condense water and heavy and medium-weight hydrocarbons. Chambers 410, 412, 414, 416, 418, 420, 422, and 424 function practically as separate temperature zones, and they are fluidically sealed from each other, except for transfer of process gases from one chamber to another through reaction tubes 430. During operation, reaction-heat-exchange chamber 412 contains a high-temperature heat-exchange medium, and interstage heat-exchange chamber 414 contains a low-temperature heat-exchange medium, as described above with reference to FIG. 2. Reactor 402 comprises a tube bundle 432 comprising reaction tubes 430. Reaction tubes 430 include first-stage tubes 434, second-stage tubes 436, third-stage tubes 438, and fourth-stage tubes 440. As described with reference to reactor 150 depicted in FIG. 2, each of tubes 430 comprises an inlet, an inlet portion, a reaction portion, an outlet portion, and an outlet. The reaction portion of a reaction tube is filled with catalyst. Typically, the inlet portion and the outlet portion are filled with blank material, which enhances heat transfer between the process gas and a heat exchange medium. Syngas feedstock enters feedstock heat-exchange chamber 410 where it is preheated and flows into and passes through first-stage reaction tubes 432. A partially reacted process stream gas is cooled in interstage heat-exchange chamber 414 to condense liquid hydrocarbon products and water. Condensed water and hydrocarbons in first interstage fluid process chamber 416 are removed from reactor vessel 404 in first liquid outlet stream 444. Process gas containing unreacted syngas enters second-stage reaction tubes 436, as indicated by arrows 446, and passes into second interstage fluid process chamber 418, where it is cooled by second-stage heat exchanger 426 to condense water and hydrocarbons. Liquid water and hydrocarbons are removed from reactor vessel 404 in second liquid outlet stream 448. Preferably, process gas containing unreacted syngas passes from interstage fluid process chamber 418 into dried-gas chamber 420 through shunt tubes 450. In dried-gas chamber 420, process gas containing unreacted syngas enters third-stage reaction tubes 438. The inlets of third-stage reaction tubes 438 are located in dried-gas chamber 420 in order to enhance condensation of water and medium-weight and heavy-weight hydrocarbons out of the process gas and to avoid carry-over of liquid into third-stage reaction tubes 438. After passing through the reaction portions of tubes 438, the process gas is cooled in the outlet portions of reaction tubes 438, which are located in interstage heat-exchange chamber 414 and first interstage fluid process chamber 416. Condensed water and hydrocarbons in third interstage fluid process chamber 422 flow out of reactor vessel 404 through the third liquid outlet stream 454. Process gas (arrows 456) containing unreacted syngas enters into and flows through fourth-stage reaction tubes 440. In exit-fluid process chamber 424, the process gas is cooled by heat exchanger 428. Condensed water and medium-weight and heavy (if present) hydrocarbons 458 exit from reactor vessel 404 through fourth liquid outlet stream 460. Process gas containing nitrogen and light hydrocarbons exits reactor 404 through gas outlet 462. As depicted in FIG. 4, tube caps 459 inhibit undesired return of liquid condensed out of a reaction stage back into reaction tubes. High-temperature heat-exchange medium flowing into and out of (arrows 460) reaction-heat-exchange chamber 412 removes most of the heat of reaction. Low-temperature heat-exchange medium flowing into and out of interstage heat-exchange chamber 414 (arrows 462) functions to cool process gas in outlet portions of reaction tubes, while preheating process gas in inlet portions of reaction tubes.

### EXAMPLE 1

A multistage compact packed-bed Fischer-Tropsch reactor in accordance with the invention was designed having the following dimensions and operating parameters, described here with reference to FIG. 2. The two-stage reactor 150, suitable for producing approximately 10 bbl/d medium-weight hydrocarbon fuel, comprises a conventional stainless-steel cylindrical reactor vessel 152 having a pressure rating of 80 atm, an inside height of 16.7 feet, and an inside diameter of 3.7 feet. A single bundle 205 comprising 415 stainless steel reactor tubes 210, 220 is supported within the reactor vessel by tube plates and end plates, as depicted in FIG. 2. Approximately 212 tubes having a length of about 12 feet are configured as first-stage reaction tubes 210; approximately 213 tubes having a length of about 14 feet are configured as second-stage reaction tubes 220. Each reactor tube has an inside diameter of 1.25 inches. A top, horizontal end plate 251 supporting second-stage tubes 220 is located about 18 inches from the top of the reactor vessel, forming an exit-fluid process chamber 168. A first tube plate 232 containing the ends of the first-stage tubes is located about 26 inches from the top of the reactor vessel, eight inches below top end plate 251, thereby defining a feedstock heat-exchange chamber 250 having a vertical length of about 8 inches. A second tube plate 234 is located about 14.2 feet from the top of the reactor vessel, 12 feet below the first tube plate, thereby defining a reaction-heat-exchange chamber 230 having a vertical length of about 12 feet. Finally, a bottom end plate 242 is located 12 inches below the second tube plate, 18 inches above the bottom of the reactor vessel, thereby defining an interstage heat-exchange chamber 240 having a vertical length of 12 inches, and an interstage fluid process chamber 156 at the bottom of the reactor having a vertical length of 18 inches.

Blank support packing 238 is packed into the bottom of the interior of the reaction tubes, corresponding to the portions of the reaction tubes located in interstage heat-exchange chamber 240. Extra blank packing is added to the second-stage tubes so that the blank packing extends about 3 inches into reaction-heat-exchange chamber 230. A cobalt-based Fischer-Tropsch catalyst 237 having a nominal catalyst particle diameter of 3 mm is then packed into the interior of the reaction tubes corresponding to the portions of the tubes enclosed by reaction-heat-exchange chamber 230. The preferred composition of catalyst 237 is 15 percent CO and 0.1 percent each of Cs, Ru, and Pt on a carbonized alumina support (97 percent alumina, 3 percent carbon). In first-stage tubes 210, about 3 inches of blank support packing is used instead of active catalyst in the first-stage inlet portion at the top of first-stage tubes 210. The total volume of the packed catalyst beds is about 1000 liters, with an approximate void space of about 50 percent. The total weight of fresh catalyst in both reaction stages is approximately 1000 kg.

Feedstock syngas comprises a mixture of about 50 percent nitrogen, 34 percent hydrogen, and 16 percent carbon monoxide by volume. Syngas at 20 atm pressure enters the feedstock heat-exchange chamber at a temperature of about 200° C., and at a flow rate of approximately 700 SCFM.

During operation, a reaction temperature of approximately 220° C. is maintained in the packed catalyst beds in the interior reaction space of the reaction tubes. Approximately 200 kilowatts (“kW”) of heat, corresponding to the heat of reaction, is removed from reaction-heat-exchange chamber 230. This is achieved by circulating conventional thermal oil at a temperature of 225° C. through reaction-heat-exchange chamber 230 at a flow rate in a range of about from 40 gallons per minute (“gpm”) to 100 gpm. The empty spaces 235 in reaction-heat-exchange chamber 230 between reaction tubes 210, 220 are filled with thermal oil. The surface area of reaction tubes 210, 220 generally necessary for a heat transfer rate of 200 kW at an oil circulation rate of 40 gpm to 100 gpm is about 60,000 in². The surface area of reaction tubes 210, 220 available for heat exchange is more than 90,000 in². Therefore, the surface area of reaction tubes 210, 220 available for heat exchange is more than sufficient to remove the heat of reaction and maintain the desired temperature. Thermal oil is circulated in the empty spaces 244 between reaction tubes in interstage heat-exchange chamber 240 at a temperature of about 35° C. and a flow rate of approximately 25 gpm. This removes approximately 15 kW of heat, thereby condensing heavy hydrocarbons (if present) and water and medium-weight hydrocarbons out of the process gas stream. The surface area of reaction tubes 210 necessary for a heat transfer rate of 15 kW at an oil circulation rate of 25 gpm is about 7,000 in². The surface area of reaction tubes 210 available for heat exchange is about 8,000 in². Condensed liquid 252 collected after the first-stage reaction at the bottom of reactor vessel 152 in interstage fluid process chamber 156 exits the reactor 150 at a flow rate of about 18 bbl/d. The condensed liquid comprises about 12 bbl/d water and about 6 bbl/d liquid hydrocarbon reaction products.

Thermal oil circulates in cooling coils 166 of exit-fluid process chamber 168 at the top of the reactor vessel to cool the process gas stream after the second-stage reaction. This removes approximately 15 kW of heat, thereby condensing and heavy hydrocarbons (if present) and water and medium-weight hydrocarbons out of the process gas stream. The thermal oil circulates at a temperature of about 35° C. and a flow rate of approximately 25 gpm. The surface area of coils 166 necessary for a heat transfer rate of 15 kW between the coils and the process gas at an oil circulation rate of 25 gpm is about 120,000 in². The surface area of coils 166 available for heat exchange is up to about 150,000 in². Condensed liquid 261 collected in exit-fluid process chamber 168 exits reactor vessel 152 at a flow rate of about 12 bbl/d. The condensed second-stage liquid comprises about 8 bbl/d water and about 4 bbl/d liquid hydrocarbon reaction products. Preferably, heated thermal oil exiting interstage fluid process chamber 240 and exit-fluid process chamber 168 is fed into reaction-heat-exchange chamber 230 as preheated high-temperature heat-exchange medium.

### EXAMPLE 2

A multistage compact packed-bed Fischer-Tropsch reactor was designed having dimensions and operating parameters similar to the reactor in Example 1, except water is utilized as a heat-exchange medium instead of thermal oil in the reaction-heat-exchange chamber, in the interstage fluid process chamber, and in the heat-exchange coils in the exit-fluid process chamber.

During operation, a reaction temperature of approximately 220° C. is maintained in the packed catalyst beds in the interior reaction space of the reaction tubes. Approximately 200 kilowatts (“kW”) of heat, corresponding to the heat of reaction, is removed from reaction-heat-exchange chamber 230. This is achieved principally using the latent heat of vaporization of water as liquid water changes into steam. The empty spaces 235 in reaction-heat-exchange chamber 230 between reaction tubes 210, 220 is filled with water, and the pressure of the reaction-heat exchange chamber is maintained at about 20 atm, which corresponds to the equilibrium vapor pressure of water at 220° C. The heat transfer coefficient between the reaction tubes and the evaporating water is several orders of magnitude greater than the heat transfer coefficient between the reaction tubes and thermal oil. Therefore, the surface area of reaction tubes 210, 220 available for heat exchange is more than sufficient to remove the heat of reaction and maintain the desired temperature. Hot steam is continuously removed from reaction-heat-exchange chamber 230, condensed and returned as liquid water at a flow rate of about 15 gallons per minute (“gpm”) to 40 gpm.

Cold heat-exchange water is circulated in the empty spaces 244 between reaction tubes in interstage heat-exchange chamber 240 at a temperature of about 35° C. and a flow rate of approximately 25 gpm. This removes approximately 15 kW of heat, thereby condensing water and medium-weight and heavy (if present) hydrocarbons out of the process gas stream. The surface area of reaction tubes 210 necessary for a heat transfer rate of 15 kW at a water circulation rate of 25 gpm is about 8,000 in². The surface area of reaction tubes 210 available for heat exchange is about 10,000 in². Condensed liquid 252 collected after the first-stage reaction at the bottom of reactor vessel 152 in interstage fluid process chamber 156 exits the reactor 150 at a flow rate of about 18 bbl/d. The condensed liquid comprises about 12 bbl/d water and about 6 bbl/d liquid hydrocarbon reaction products.

Cooling water circulates in cooling coils 166 of exit-fluid process chamber 168 at the top of the reactor vessel to cool the process gas stream after the second-stage reaction. This removes approximately 15 kW of heat, thereby condensing heavy hydrocarbons (if present) and water and medium-weight hydrocarbons out of the process gas stream. The cooling water circulates at a temperature of about 35° C. and a flow rate of approximately 25 gpm. The surface area of coils 166 necessary for a heat transfer rate of 15 kW at a water circulation rate of 25 gpm is about 120,000 in². The surface area of coils 166 available for heat exchange is up to about 150,000 in². Condensed liquid 261 collected in exit-fluid process chamber 168 exits reactor vessel 152 at a flow rate of about 12 bbl/d. The condensed second-stage liquid comprises about 8 bbl/d water and about 4 bbl/d liquid hydrocarbon reaction products. Preferably, heated cooling water exiting interstage fluid process chamber 240 and exit-fluid process chamber 168 is fed into reaction-heat-exchange chamber 230 as a preheated high-temperature heat-exchange medium.

A multistage reactor and a method in accordance with the invention provide for interstage condensation and removal of product hydrocarbons out of the process gas stream containing reactant syngas. This increases the overall conversion of the Fischer-Tropsch process. Embodiments in accordance with the invention also provide interstage condensation and removal of water out of the reactant gas stream, resulting in improved catalyst performance, which also increases reaction yields. A compact multistage reactor in accordance with the invention physically combines a plurality of catalytic reaction stages and several related heat-exchange operations within a single reactor vessel. This improves thermal efficiency of a Fischer-Tropsch process relative to processes conducted using conventional designs and equipment, and reduces operating costs. Significantly, a compact design in accordance with the invention reduces equipment costs and associated capital investment costs. A compact design allows a reactor in accordance with the invention to be transported and installed more easily and less expensively. In addition, a compact reactor occupies less physical space, providing an advantage when larger space is unavailable or expensive, for example, on an offshore oil platform or in an urban setting.

It is evident that those skilled in the art may now make numerous uses and modifications of the specific embodiments described, without departing from the inventive concepts. It is also evident that the particular components and processes described and recited may, in some instances, be conducted and performed in a different order; or equivalent structures and processes may be substituted for the structures and processes described. For example, in certain embodiments, the function of the interstage heat-exchange chamber and the interstage fluid-process chamber are combined in a single, fluid-process chamber comprising a conventional heat exchanger to effect condensation. Or, for example, the feedstock heat-exchange chamber is eliminated and feedstock flows into the first-stage tubes by a manifold system. Since certain changes may be made in the above apparatus and methods without departing from the scope of the invention, it is intended that all subject matter contained in the above description or shown in the accompanying drawing be interpreted as illustrative and not in a limiting sense. Consequently, the invention is to be construed as embracing each and every novel feature and novel combination of features present in or inherently possessed by the systems, methods and compositions described in the claims below and by their equivalents.

1. A multistage compact packed-bed Fischer-Tropsch reactor, comprising:
a reactor vessel;
a first-stage tube disposed in said reactor vessel, said first-stage tube defining a first interior reaction space;
a second-stage tube disposed in said reactor vessel, said second-stage tube defining a second interior reaction space;
an interstage fluid process chamber disposed in said reactor vessel; and
a first heat exchanger disposed in said reactor vessel.

2. A reactor as in claim 1, further comprising a liquid-removal outlet in said interstage fluid process chamber.

3. A reactor as in claim 1 wherein said first heat exchanger is disposed in said interstage fluid process chamber.

4. A reactor as in claim 1 wherein said first-stage tube includes a first stage outlet in said interstage fluid process chamber, and said second-stage tube includes a second-stage inlet in said interstage fluid process chamber.

5. A reactor as in claim 1, further comprising a baffle disposed in said interstage fluid process chamber.

6. A reactor as in claim 1, further comprising an interstage syngas inlet in fluidic communication with said interstage fluid process chamber.

7. A reactor as in claim 1, further comprising an exit-fluid process chamber disposed in said reactor vessel, said second-stage tube including a second-stage outlet in said exit-fluid process chamber.

8. A reactor as in claim 7, further comprising a second heat exchanger disposed in said exit-fluid process chamber for condensing hydrocarbons and water from a process gas.

9. A reactor as in claim 7, further comprising:
a process gas outlet in said exit-fluid process chamber; and
a liquid-removal outlet in said exit-fluid process chamber.

10. A reactor as in claim 7, further comprising a baffle disposed in said exit fluid process chamber.

11. A reactor as in claim 1, further comprising:
a reaction-heat-exchange chamber disposed in said reactor vessel;
a first-stage reaction portion of said first-stage tube being located in said reaction-heat-exchange chamber, and a second-stage reaction portion of said second-stage tube being located in said reaction-heat-exchange chamber.

12. A reactor as in claim 11, further comprising:
a packed bed of Fischer-Tropsch catalyst disposed within said first-stage reaction portion of said first-stage tube; and
a packed bed of Fischer-Tropsch catalyst disposed within said second-stage reaction portion of said second-stage tube.

13. A reactor as in claim 12, further comprising a fluid heat-exchange medium disposed in said reaction-heat-exchange chamber, said heat-exchange medium being in thermal contact with an outer surface of said reaction portions.

14. A reactor as in claim 13 wherein said fluid heat-exchange medium is selected from a group consisting of water and thermal oil.

15. A reactor as in claim 14 wherein said heat-exchange medium comprises water, and further comprising a pressure controller for maintaining a pressure in said reaction-heat-exchange chamber exterior to said tubes.

16. A reactor as in claim 11 wherein said first heat exchanger comprises:
an interstage heat-exchange chamber disposed in said reactor vessel;
a first-stage outlet portion of said first-stage tube, said first-stage outlet portion being located in said interstage heat-exchange chamber; and
a second-stage inlet portion of said second-stage tube, said second-stage inlet portion being located in said interstage heat-exchange chamber.

17. A reactor as in claim 16, further comprising a heat-exchange medium disposed in said interstage heat-exchange chamber, said heat-exchange medium in thermal contact with an outside surface of said outlet portion and with an outside surface of said inlet portion.

18. A reactor as in claim 17 wherein said fluid heat-exchange medium is selected from a group consisting of water and thermal oil.

19. A reactor as in claim 16 wherein said first-stage outlet portion and said second-stage inlet portion do not contain catalyst.

20. A reactor as in claim 19 wherein said first-stage outlet portion and said second-stage inlet portion contain blank packing.

21. A reactor as in claim 11 wherein said first-stage tube comprises a first-stage inlet portion disposed at least partly in said reaction-heat-exchange chamber.

22. A reactor as in claim 21 wherein said first-stage inlet portion does not contain catalyst.

23. A reactor as in claim 22 wherein said first-stage inlet portion comprises blank packing.

24. A reactor as in claim 1, further comprising:
a feedstock heat-exchange chamber disposed in said reactor vessel;
wherein said first-stage tube comprises a first-stage inlet in fluidic communication with said feedstock heat-exchange chamber, and said feedstock heat-exchange chamber comprises at least part of a second-stage outlet portion of said second-stage tube.

25. A reactor as in claim 1, further comprising:
a plurality of first-stage tubes; and
a plurality of second-stage tubes.

26. A reactor as in claim 25 wherein a plurality of first-stage tubes and a plurality of second-stage tubes are included in a tube bundle.

27. A reactor as in claim 1, further comprising:
a plurality of sequential reaction stages, each reaction stage comprising at least one reaction tube disposed in said reactor vessel, each reaction tube defining an interior reaction space; and
a plurality of interstage fluid process chambers.

28. A reactor as in claim 27, further comprising a plurality of interstage heat-exchange chambers disposed in said reactor vessel.

29. A method of conducting a Fischer-Tropsch reaction in a multistage compact packed-bed reactor, comprising:
flowing process gas containing inlet syngas through a first catalyst bed, said first catalyst bed disposed in an interior reaction space of a first-stage reaction tube located in a reactor vessel, to convert syngas into hydrocarbons;
then first-stage-cooling said process gas within said reactor vessel to condense hydrocarbons and water from partially reacted process gas;
then flowing said partially reacted process gas into a second catalyst bed, said second catalyst bed disposed in an interior reaction space of a second-stage reaction tube located in said reactor vessel, to convert syngas into hydrocarbons;
then second-stage-cooling said process gas within said reactor vessel to condense hydrocarbons and water from said process gas.

30. A method as in claim 29, further comprising removing liquid hydrocarbons and liquid water from said reactor vessel after said first-stage cooling.

31. A method as in claim 29 wherein said first-stage cooling comprises contacting an exterior surface of a first-stage outlet portion of said first-stage reaction tube with a heat-exchange medium.

32. A method as in claim 29 wherein said first-stage cooling comprises flowing said process gas through a heat exchanger disposed in an interstage fluid processing chamber.

33. A method as in claim 29 wherein said first-stage cooling and said second-stage cooling are conducted at a temperature in a range of about from 20° C. to 40° C.

34. A method as in claim 29, further comprising removing liquid hydrocarbons and liquid water from said reactor vessel after said second-stage cooling.

35. A method as in claim 29, further comprising maintaining a pressure in said catalyst beds in a range of about from 10 atmospheres to 20 atmospheres.

36. A method as in claim 29, further comprising maintaining a temperature of said first catalyst bed and said second catalyst bed.

37. A method as in claim 36 wherein said maintaining a temperature of said catalyst beds comprises maintaining a reaction temperature in a range of about from 150° C. to 280° C.

38. A method as in claim 36 wherein said maintaining a temperature of said catalyst beds comprises contacting an exterior surface of said reaction tubes with a high-temperature heat-exchange medium.

39. A method as in claim 38 wherein said maintaining a temperature of said catalyst beds comprises contacting an exterior surface of said reaction tubes with a thermal oil.

40. A method as in claim 38 wherein said maintaining a temperature of said catalyst beds comprises providing liquid water in a reaction-heat-exchange chamber and maintaining a pressure in said reaction-heat-exchange chamber such that said liquid water boils at a desired reaction temperature.

41. A method as in claim 29, further comprising first-stage-preheating said process gas before flowing said process gas through said first catalyst bed.

42. A method as in claim 41 wherein said first-stage-preheating comprises contacting an exterior surface of a first-stage inlet portion of said first-stage reaction tube with said high-temperature heat-exchange medium, thereby transferring internal system heat to said first-stage inlet portion.

43. A method as in claim 29, further comprising second-stage-preheating said process gas before flowing said process gas through said second catalyst bed.

44. A method as in claim 43 wherein said second-stage-preheating comprises contacting an exterior surface of a first-stage outlet portion of said first-stage reaction tube with a heat-exchange medium, and contacting an exterior surface of a second-stage inlet portion of a second-stage reaction tube with said heat-exchange medium, thereby transferring internal system heat from said first-stage reaction tube to said second-stage reaction tube.

45. A multistage compact chemical reactor, comprising:
a reactor vessel;
a first-stage tube disposed in said reactor vessel, said first-stage tube defining an interior reaction space;
a second-stage tube disposed in said reactor vessel, said second-stage tube defining an interior reaction space;
a heat exchanger disposed in said reactor vessel;
an interstage fluid process chamber disposed in said reactor vessel; and
a fluid removal outlet in said interstage fluid process chamber.

46. A multistage compact chemical reactor as in claim 45, further comprising an exit-fluid process chamber disposed in said reactor vessel.

47. A multistage compact chemical reactor as in claim 45, further comprising:
an interstage heat-exchange chamber disposed in said reactor vessel;
a first-stage outlet portion of said first-stage tube located in said interstage heat-exchange chamber; and
a second-stage inlet portion of said second-stage tube located at least partly in said interstage heat-exchange chamber.

48. A multistage compact chemical reactor as in claim 45, further comprising a feedstock heat-exchange chamber disposed in said reactor vessel.

49. A multistage compact chemical reactor as in claim 45, further comprising:
a plurality of first-stage tubes; and
a plurality of second-stage tubes.
